(19) United States
(12) Patent Application Publication
(10) Pub. No.: US 2022/0054368 A1
(43) Pub. Date: Feb. 24, 2022

(54) Redispersible Three-Layer Cosmetic

(71) Applicant: L V M H Recherche, SAINT JEAN DE BRAYE (FR)

(72) Inventors: Miyako KITAMURA, Tokyo (JP); Mai OZAWA, Tokyo (JP); Takayoshi SAKODA, Tokyo (JP)

(73) Assignee: L V M H Recherche, SAINT JEAN DE BRAYE (FR)

(21) Appl. No.: 17/415,282

(22) PCT Filed: Dec. 19, 2018
(86) PCT No.: PCT/IB2018/001600

§ 371 Date: Jun. 17, 2021

(51) Int. Cl. A61K 8/03; A61K 8/73; A61K 8/92; A61Q 19/00

(52) U.S. Cl. CPC A61K 8/03; A61K 8/73; A61K 2800/412; A61Q 19/007; A61K 8/92

(57) ABSTRACT

A redispersible three-layer cosmetic comprising an aqueous phase, an oil phase a polysaccharide gel particles having a mean particle size of 0.1 to 1000 μm.

### TECHNICAL FIELD

The present invention relates to a redispersible three-layer cosmetic.

### BACKGROUND ART

Multilayer cosmetics are composed of multiple layers (for example, a powder layer and/or an oil layer and an aqueous layer). Many different technologies for multilayer cosmetics continue to be developed because such multilayer cosmetics provide excellent aesthetic appearance and are very attractive for consumers.

Japanese Unexamined Patent Publication No. 2013-177366 (equivalent EP2810639B1), for example, discloses a redispersible powder-dispersed cosmetic comprising (A) succinic acid and/or its salt, (B) bentonite and (C) a hydrophilic surfactant. But in a multilayer cosmetic composed of an aqueous layer and a powder layer comprising specific powders such as bentonite, cellulose and a synthetic polymer, it is often the case that the components become adsorbed onto the skin due to the moistness of the powder layer, impacting the long-lasting moisturizing effect searched by the consumers.

Also, Japanese Unexamined Patent Publication No. 2007-126394 discloses a multilayer cosmetic comprising a copolymer obtained by polymerizing a specific polyethylene oxide macromonomer, a hydrophobic monomer and a crosslinkable monomer, with 5 to 40 mass % of a liquid oil, and with the surfactant content being within a specified range. Japanese Unexamined Patent Publication No. 2014-208634 discloses a multilayer cosmetic comprising the following components (a) to (d): (a) a nonvolatile hydrocarbon oil having a viscosity of 30 to 400 mm²/s at 25° C., (b) a silicone oil having a viscosity of 30 to 400 mm²/s at 25° C., (c) water and (d) an ester of isostearic acid and polyglycerin.

But such a multilayer cosmetic having an aqueous layer and an oil layer tends to produce an oily sticky feel, impacting the smoothness searched by the consumers.

So there is still a need for providing a multi-layer cosmetic that is improved in terms of both long-lasting moisturizing effect and smoothness.

Research carried out by the present inventors has led to the discovery of a novel redispersible three-layer cosmetic composed of specific gel particles, an aqueous phase and an oil, and the three-layer cosmetic has been demonstrated to exhibit improved long-lasting moisturizing effect and smoothness.

### SUMMARY OF THE INVENTION

The invention relates to a redispersible three-layer cosmetic comprising an aqueous phase, an oil and a polysaccharide gel particle having a mean particle size of 0.1 to 1000 μm.

The expression ‘redispersible three-layer cosmetic’ according to the invention, means that the composition provides a good redispersibility of polysaccharide gel particles (bottom layer) in the aqueous phase and/or oil phase when shaken for use while being able to give a clear supernatant (aqueous phase and oil phase) when not-in-use. So, settling and dispersion of the gel particle in the aqueous phase and/or oil phase takes place repeatedly through cycles of standing at rest (not-in-use) and agitation (shaken).
The term ‘three-layer’ cosmetic according to the invention, means that the composition presents three visually distinct layers, when it stands at rest (not-in-use), ie an upper layer comprising the oil phase, a intermediate layer comprising the aqueous phase and a bottom layer comprising the polysaccharide gel particles. The redispersible three-layer cosmetic of the invention exhibits improved long-lasting moisturizing effect and smoothness.

The polysaccharide may be agar. In this case, as a gel particle, a pulverized form of an agar gel may be used and the agar gel is obtained by gelating the agar swelled with a gel-forming aqueous phase (second layer).

The viscosity of the three-layer cosmetic may be 1 cP to 200 cP at 25° C.

The aqueous phase may comprise a polyol or a mono-alcohol. The aqueous phase may also comprise an aryloxyalkanol such as phenoxyethanol. The oil in the three-layer cosmetic may comprise two or more selected from the group consisting of polar oils, non-polar oils and silicone oils. This will result in even more excellent long-lasting moisturizing effect and smoothness.

### Advantageous Effects of Invention

With the invention it is possible to provide a redispersible three-layer cosmetic that is improved in terms of both long-lasting moisturizing effect and smoothness.

### DETAILED DESCRIPTION OF THE INVENTION

Embodiments of the invention will now be described. However, the present invention is not limited to the embodiments described below.

The redispersible three-layer cosmetic of this embodiment contains an aqueous phase (second layer, ie intermediate layer), an oil phase (first layer, ie upper layer) and polysaccharide gel particles having a mean particle size of 0.1 to 1000 μm (third layer, ie bottom layer). As used herein, the term “three-layer cosmetic” refers to a cosmetic having three visually distinct layers when not-in-use, ie a plurality of gel particles separate from the aqueous phase and oil phase.

When the three-layer cosmetic of this embodiment undergoes a cycle of standing at rest (not-in-use) and agitation (shaken), settling and dispersion of the gel particle takes place in the aqueous phase and/or oil. Specifically, during settling (for example, when approximately 100 mL is housed in a container with a capacity of 120 mL and allowed to stand for one day or longer), the gel particles settle in the aqueous phase and/or oil phase, so that the three-layer cosmetic consists of a bottom layer formed by a plurality of gel particle aggregates, an intermediate layer composed of the aqueous phase over the bottom layer, and an upper layer composed of the oil phase. The gel particles are composed of components such that, under usual storage temperature (for example, room temperature, or 5 to 30° C.) and storage time (for example, 3 years) conditions for a three-layer cosmetic, they do not elute into the aqueous phase and lose their form, and the particles do not adhere together and form aggregates (masses). The aqueous phase may also be present between the gel particles. When agitated (shaken), however, the gel particles become dispersed in the aqueous phase and/or oil phase, and therefore the three-layer cosmetic comprises the aqueous phase, the oil phase, and the gel particles that have become dispersed in the aqueous phase and/or oil phase.

### Aqueous Phase (Second Layer, ie Intermediate Layer)

The aqueous phase may consist of water alone or it may comprise other water-soluble components in water. Water-soluble components are components that dissolve to at least 0.1 g per 100 g of water.

Water-soluble components comprise polyols, mono-alcohols (excluding those qualifying as preservatives), preservatives, perfumes, salting-out agents, pH regulators, surfactants, whitening agents, anti-inflammatory agents, colorants, and non-gelating polysaccharides (for example, xanthan gum, sodium hyaluronate, gum arabic, xanthan gum, guar gum and succinoglycan).

The valency of a polyol may be 2 to 4, or 2 to 3, for example. In other words, the polyol may comprise a diol and/or triol. Examples of polyols comprise diols such as butylene glycol (1,3-butylene glycol), pentylene glycol, propanediol, dipropylene glycol, polyalkylene glycols (such as polyethylene glycol), and triols such as glycerin. Such polyols may be used alone or in combinations of two or more.

When the aqueous phase comprises or consists of a polyol, the total polyol content may be 1 to 30 mass %, 5 to 20 mass % or 7 to 15 mass %, based on the total mass of the three-layer cosmetic. The term ‘mass %’ is also referred as % by weight.

Mono-alcohols (C1-C5) comprise ethanol, propanol, isopropanol and butanol. In a particular embodiment, the mono-alcohol is ethanol. These mono-alcohols may also be used alone or in combinations of two or more. When the aqueous phase comprises or consists of a mono-alcohol, the total monool content may be 1 to 20 mass %, 2 to 15 mass % or 3 to 10 mass % based on the total mass of the three-layer cosmetic.

Preservatives comprise aryloxyalkanols such as phenoxyethanol, and ethylenediaminetetraacetic acid (EDTA) disodium and ethylenediaminetetraacetic acid (EDTA) tetrasodium. The aqueous phase may comprise an aryloxyalkanol as the preservative, but it preferably comprises phenoxyethanol. Such preservatives may also be used alone or in combinations of two or more. When the aqueous phase comprises a preservative, the preservative content may be 0.01 to 5 mass %, 0.01 to 2 mass %, 0.1 to 2 mass %, 0.5 to 2 mass % or 0.7 to 2 mass %, based on the total mass of the three-layer cosmetic.

Salting-out agents comprise sodium chloride, potassium chloride, calcium chloride, sodium sulfate and magnesium sulfate. When the aqueous phase comprises a salting-out agent, the total salting-out agent content may be 0.1 to 10 mass %, 0.5 to 5 mass % or 0.7 to 3 mass %, based on the total mass of the three-layer cosmetic.

The aqueous phase content may be 70.0 to 95.0 mass % or 75.0 to 90.0 mass %, based on the total mass of the three-layer cosmetic.

### Oil Phase (First Layer, ie Upper Layer)

The oil phase generally comprises a liquid oil (for example, an oil that is liquid at room temperature, i.e. about 5 to 30° C.). The oil may have a lower specific gravity than water. Oils comprise polar oils, non-polar oils and silicone oils. The oil may be either a volatile oil or a non-volatile oil.

Oils comprise polar oils such as isononyl isononanoate, ethylhexyl isononanoate, isotridecyl isononanoate, cetearyl isononanoate, cetearyl ethylhexanoate, ethyl oleate, neopentyl glycol dicaprate, neopentyl glycol diethylhexanoate, alkyl benzoate (C12-15), diisostearyl malate, di(isostearyl/phytostearyl) dilinolate dimer, di(phytostearyl/octyldodecyl) lauroylglutamate, triethylhexanoin glyceryl tri(2-ethylhexanoate)), glyceryl tri(caprylate/caprate), polyglyceryl-2 triisostearate, pentaerythrityl tetraisostearate, jojoba (Simmondsia chinensis) seed oil, olive fruit oil, sunflower seed oil, meadowfoam oil, rice bran oil, safflower oil and tocopherol, and nonpolar solvents such as isododecane, isohexadecane, (C9-12)alkane, hydrogenated polyisobutene, hydrogenated polydecene, olefin oligomer and squalane. Preferred polar oils comprise isononyl isononanoate, triethylhexanoin and jojoba seed oil. Preferred non-polar oils comprises squalane, isododecane and hydrogenated polyisobutene.

Silicone oils may also be used as oils. Silicone oils may be either volatile silicone oils or non-volatile silicone oils. Silicone oils comprise dimethylpolysiloxane (dimethicone), methyltrimethicone, cyclopentasiloxane and phenyltrimethicone. Preferred silicone oils comprise methyltrimethicone, cyclopentasiloxane and dimethicone.

Such oils may be used alone or in combinations of two or more. From the viewpoint of even more improved long-lasting moisturizing effect, the oil may further comprise polar oils. From the viewpoint of easily obtaining three-layer cosmetic having a clear interface, the oil may further comprise non-polar oils and/or silicone oils. The oil may consist of either a polar solvent alone or a nonpolar solvent alone, or it may be a mixed solvent comprising two or more selected from the group consisting of a polar solvent, a nonpolar solvent and a silicone oil.

From the viewpoint of even more excellent long-lasting moisturizing effect and smoothness, the oil may further comprise at least one selected from the group consisting of polar oils, non-polar oils and silicone oils. The oil may comprise at least one selected from the group consisting of isononyl isononanoate, isododecane, methyltrimethicone, cyclopentasiloxane, dimethicone, triethylhexanoin, squalane, jojoba seed oil and hydrogenated polyisobutene. From the viewpoint of yet even more excellent long-lasting moisturizing effect and smoothness, the oil may comprise isononyl isononanoate or hydrogenated polyisobutene, or a polar solvent and a nonpolar solvent, or at least one polar solvents selected from the group consisting of triethylhexanoin and isononyl isononanoate and at least one nonpolar solvents selected from the group consisting of squalane, isododecane and hydrogenated polyisobutene, or triethylhexanoin and squalane, or isononyl isononanoate and at least one nonpolar solvents selected from the group consisting of isododecane and hydrogenated polyisobutene. When the oil comprises a polar solvent and a nonpolar solvent, the content ratio of the polar solvent and nonpolar solvent by mass (polar solvent:nonpolar solvent) may be 1:9 to 9:1, 1:5 to 5:1 or 1:3 to 3:1, for example.

The oil content may be 1.0 to 40 mass %, 5.0 to 30.0 mass % or 10.0 to 20.0 mass %, based on the total mass of the three-layer cosmetic.

### Polysaccharide Gel Particles (Third Layer, ie Bottom Layer)

The gel particle comprises a gellable polysaccharide, and a solvent in the third layer for formation of the gel (a gel-forming aqueous phase of the third layer). Examples of gellable polysaccharides (hereunder also referred to simply as “polysaccharides”) comprise agar, carrageenan (examples of which are kappa-carrageenan and iota-carrageenan), gellan gum, sodium alginate, tamarind gum, mannan and locust bean gum, and mixtures thereof. These polysaccharides may likewise be used alone or in combinations of two or more. The polysaccharide is preferably at least one selected from the group consisting of agar, carrageenan and gellan gum, and more preferably agar. The gel-forming aqueous phase of the third phase may employ any of the components mentioned above for the aqueous phase (second layer). The components used in the gel-forming aqueous phase (third layer) may be the same as in the aqueous phase described above (second layer), or different ones. In a particular embodiment, the solvent used for formation of the polysaccharide gel is water.

A gel particle can be obtained by the following method, as an example. First, the gellable polysaccharide is swelled with the aqueous phase of the third layer (a gel-forming aqueous phase of the third layer) by heating if necessary. The temperature for swelling the polysaccharide may be 70 to 100° C. or 80 to 90° C., for example. The polysaccharide may be swelled while being stirred under conditions of 1000 to 5000 rpm, for example. The swelled polysaccharide is allowed to stand for cooling, and then to be pulverized, or the swelled polysaccharide is cooled while being stirred, to obtain polysaccharide a gel particle having a mean particle size of 0.1 to 1000 μm. The pulverization can be carried out using a homogenizer, Disper mixer or blender, for example. The pulverization may alternatively be carried out by stirring at 5000 rpm to 20,000 rpm. A gelling agent (such as calcium chloride or potassium chloride) may also be used during the process of producing the gel particle. When a gelling agent is used for gelation of the polysaccharide, it is preferred to select one such that the jelly strength of the polysaccharide is between 500 g/cm² and 1000 g/cm² at a polysaccharide concentration of 1.5 mass %, for example. The gel particles do not have a thickening effect that is effective enough to be used as a thickening agent for cosmetics.

When agar is used as a polysaccharide, as a gel particle a pulverized form of an agar gel may be used. As an agar gel, agar swelled with the gel-forming aqueous phase of the third layer and gelated may be used. The pulverized form of an agar gel is obtained by allowing the swelled agar to stand for cooling then pulverizing the same or by cooling while stirring the swelled agar.

When the polysaccharide is at least one selected from the group consisting of carrageenan, gellan gum, sodium alginate, tamarind gum, mannan and locust bean gum, the gel particle may be a pulverized form of the polysaccharide gel, a polysaccharide gel obtained by gelating the polysaccharide swelled with the gel-forming aqueous phase of the third layer. In this case, gelation may be carried out by adding a gelling agent.

The mean particle size of the gel particle may be 0.1 μm or greater, 1 μm or greater, 5 μm or greater, 10 μm or greater, 50 μm or greater, 60 μm or greater, 70 μm or greater or 80 μm or greater, to no greater than 1000 μm, no greater than 500 μm, no greater than 400 μm, no greater than 300 μm, no greater than 250 μm, no greater than 200 μm, no greater than 150 μm or no greater than 120 μm. In other words, the mean particle size of the gel particle may be 0.1 to 1000 μm, 1 to 500 μm, 5 to 400 μm, 10 to 300 μm, 50 to 250 μm, 60 to 200 μm, 70 to 150 μm or 80 to 120 μm. The term “mean particle size” as used herein is defined as the value measured using a Laser scattering particle size distribution analyzer. The mean particle size of the gel particle can be adjusted to within such ranges by altering the conditions under which the polysaccharide gel particle are produced (such as the pulverizing conditions).

When the polysaccharide is agar, the jelly strength (or gelation strength) of the polysaccharide may be 500 g/cm² or greater, 600 g/cm² or greater, 650 g/cm² or greater or 700 g/cm² or greater, to no greater than 1000 g/cm², no greater than 900 g/cm², no greater than 800 g/cm² or no greater than 750 g/cm², with a polysaccharide concentration of 1.5 mass %. From the viewpoint of superior smoothness, when the polysaccharide is agar its jelly strength may be from 500 g/cm² to 1000 g/cm², from 600 g/cm² to 900 g/cm², from 650 g/cm² to 800 g/cm² or from 700 g/cm² to 800 g/cm², at a polysaccharide concentration of 1.5 mass %.

The jelly strength (or gelation strength) is measured as a gel strength for an aqueous solution having a 1.5% polysaccharide concentration. In other words, for the jelly strength measurement, the polysaccharide is weighed accurately, and deionized water is added thereto, thereby causing the polysaccharide to sufficiently absorbs water. Subsequently, warm deionized water is added thereto to adjust the content, which is then subjected to a hot water bath to cause dissolution by heat. In order to make up water evaporating by heating, deionized water is used as a supplement to adjust the content, and the solution is caused to flow into a glass container in which a tape is wound around the upper portion thereof. The container is left to cool at room temperature, and then capped and left in a constant temperature chamber at 20° C. overnight. The tape is peeled off from the glass container, and then jelly around the periphery of the container that sticks out of the container is cut with a cutter and discarded. The strength of the cut surface of the obtained jelly is measured using a texture analyzer or the like. That is, a cylindrical plunger having an area of 1 cm² is mounted on the cut surface, and the sample stage is moved at an appropriate lifting rate. In this manner, a force applied until the jelly breaks can be measured.

From the viewpoint of allowing the gel particle to settle more easily, the weight-average molecular weight of the polysaccharide may be 150,000 or greater, 200,000 or greater, 250,000 or greater or 300,000 or greater, and from the viewpoint of superior smoothness (minimally rough surface), it is preferably no greater than 500,000, no greater than 450,000, no greater than 400,000 or no greater than 350,000. The weight-average molecular weight of the polysaccharide may be from 150,000 to 500,000, from 150,000 to 450,000, from 150,000 to 400,000, from 150,000 to 350,000, from 200,000 to 500,000, from 200,000 to 450,000, from 200,000 to 400,000, from 200,000 to 350,000, from 250,000 to 500,000, from 250,000 to 450,000, from 250,000 to 400,000, from 250,000 to 350,000, from 300,000 to 500,000, from 300,000 to 450,000, from 300,000 to 400,000 or from 300,000 to 350,000.

The weight-average molecular weight of the polysaccharide can be measured by HPLC gel permeation chromatography. For example, after dissolving the polysaccharide in distilled water at 95 to 97° C., the solution is cooled to 50° C. to obtain a measuring sample, and the gel permeation chromatography measurement is conducted using this sample. One example of a liquid chromatography apparatus is LC-10AT VP or RID-10A by Shimadzu Corp., with a differential refractometer as the detector, TOSOH TSK-GEL for HPLC or TSK-GEL GMPWXL by Tosoh Corp. as the column and 0.1 M sodium nitrate as the developing solvent, and the measurement being conducted at a constant temperature. The weight-average molecular weight of agar is determined using pullulan of known molecular weight (Shodex STANDARD P-82, for example) as the standard sample. The standard sample is dissolved in distilled water, and measurement is performed by HPLC gel permeation chromatography under the same conditions.

The polysaccharide content may be 0.01 mass % or greater, 0.05 mass % or greater, 0.10 mass % or greater, 0.20 mass % or greater, 0.30 mass % or greater or 0.40 mass % or greater, to no greater than 2.0 mass %, no greater than 1.0 mass %, no greater than 0.8 mass % or no greater than 0.6 mass %, based on the total mass of the three-layer cosmetic. In other words, the polysaccharide content may be from 0.05 to 2.0 mass %, in particular from 0.10 to 1.0 mass %, or from 0.20 to 0.8 mass % or even from 0.40 to 0.6 mass %, based on the total mass of the three-layer cosmetic.

The polysaccharide gel particles content may be 5 mass % or greater, 10 mass % or greater or 15 mass % or greater, to no greater than 50 mass %, no greater than 30 mass % or no greater than 25 mass %, based on the total mass of the three-layer cosmetic. The polysaccharide gel particles content may be from 5 to 50 mass %, in particular from 10 to 30 mass % or even from 15 to 25 mass %, based on the total mass of the three-layer cosmetic.

The three-layer cosmetic may also comprise a perfume, for example, in addition to the components mentioned above. The perfume content may be set as appropriate depending on the type of perfume. For example, the perfume content may be from 0.001 to 1.0 mass % or in particular 0.01 to 0.5 mass %, based on the total mass of the three-layer cosmetic. The perfume may be dissolved in either or both the oil phase and aqueous phase.

The viscosity of the three-layer cosmetic may be no higher than 200 cP, no higher than 100 cP, no higher than 50 cP or lower than 10 cP, and it may be 1 cP or higher, or 5 cP or higher, for example, at 25° C. That is, the viscosity of the three-layer cosmetic may be 1 cP to 200 cP, 1 cP to 100 cP, 1 cP to 50 cP, 1 cP to less than 10 cP, 5 cP to 200 cP, 5 cP to 100 cP, 5 cP to 50 cP or 5 cP to less than 10 cP, at 25° C.

The viscosity of the three-layer cosmetic may be measured as the shear viscosity using a rotating viscometer (Rheolab QC by Anton Paar GmbH), under conditions of 100 rpm, 25° C. A viscosity in this range will improve the stability of the three-layer cosmetic and give it an excellent feel during use.

The redispersible three-layer cosmetic can be obtained, for example, by mixing (shaking) and stirring the aqueous phase of the second layer, the oil phase of the first layer and the gel particles of the third layer.

The redispersible three-layer cosmetic of this embodiment can be suitably used for a cosmetic water product, cleansing lotion, face cleanser, essence, makeup base, lotion mist, sunscreen or the like.

The present invention also relates to a cosmetic process for caring for and/or making-up keratinic materials comprising the application onto keratinic materials, in particular onto skin, of the redispersible three-layer cosmetic as defined in the invention.
By ‘keratinic materials’, it means skin and/or lips, preferably skin.
The redispersible three-layer cosmetic is generally shaken before use.
In particular, the redispersible three-layer cosmetic of the invention advantageously provides a long-lasting moisturizing effect and smoothness onto keratinic materials, in particular onto skin, on which it is applied.

### EXAMPLES

The invention will now be illustrated by examples, with the understanding that the invention is not meant to be limited to these examples. Unless contrary indication, the % are mass % also referred as % by weight of total weight of the composition.

The following polysaccharides were prepared.
Agar 1 (trade name: Ina Agar CS-7, Ina Food Industry Co., Ltd., INCI name: AGAR, jelly strength (1.5 mass % concentration): 730±20 (g/cm³), weight-average molecular weight: 300,000).
Agar 2 (trade name: Ina Agar CS-310, Ina Food Industry Co., Ltd., INCI name: AGAR, jelly strength (1.5 mass % concentration): 100±50 (g/cm³), weight-average molecular weight: 100,000).
Agar 3 (trade name: Ina Agar CS-33, Ina Food Industry Co., Ltd., INCI name: AGAR, jelly strength (1.5 mass % concentration): 850±50 (g/cm³), weight-average molecular weight: 700,000).
Carrageenan (trade name: GENUGEL®, SWG-J type: kappa, CPKelco Co., Ltd, INCI name: CARRAGEENAN (Kappa)).
Gellan gum (trade name: KELCOGEL, CPKelco Co., Ltd, INCI name: GELLAN GUM (LA type)).

The jelly strength of agar and the weight-average molecular weight are the values measured by the above-mentioned method.

Polysaccharide gel particles were prepared from the polysaccharide by the following method. First, water (the gel-forming aqueous phase of the third layer) and the polysaccharide were combined, and while the combination was stirred at 90° C., 3000 rpm, the polysaccharide was swelled. When the agar was used as the polysaccharide, the swelled polysaccharide was allowed to stand for cooling to obtain an agar gel (polysaccharide concentration: 4 mass %). The gelling agent that was added and mixed was potassium chloride for kappa carrageenan and calcium chloride for gellan gum to obtain a gel (polysaccharide concentration: 4 mass %). Water (the gel-forming aqueous phase of the third layer) was added to the gel which was then pulverized with a Waring blender at 18,000 rpm for 2 minutes to prepare polysaccharide gel particles (polysaccharide concentration: 2 mass %).

Aqueous phases (second layer) and oil phases (first layer) having the compositions listed in Tables 1 to 3 were added to the polysaccharide gel particles obtained by the method described above, and the mixtures were stirred to prepare three-layer cosmetics for the Examples. The polysaccharide contents of the three-layer cosmetics of the Examples were the amounts listed in Tables 1 to 3 with respect to the total masses of the three-layer cosmetics.

Bentonite and kaolin (powder phase) were also mixed and dispersed with an aqueous phase and oil phase having the composition listed in Table 1 to prepare a three-layer cosmetic for Comparative Example 1 to 2. Cosmetics having the compositions listed in Tables 1 and 2 were also prepared as cosmetics for Comparative Examples 3 and 5.

Agar (trade name: Ina Agar CS-7, Ina Food Industry Co., Ltd.) and water were combined, and while the combination was stirred at 90° C., 3000 rpm, the agar was swelled. An aqueous phase and oil phase having the composition listed in Table 1 were added to the swelled agar and the mixture was stirred to prepare a cosmetic for Comparative Example 6.

In Examples 1 to 4 and 8 to 21, the mean particle sizes of the agar gel particles were 100 μm. In Example 5, the mean particle size of the carrageenan gel particles was 100 μm, and in Example 6 the mean particle size of the gellan gum gel particles was 100 μm. The mean particle size of the gel particles was measured using a Laser scattering particle size distribution analyzer.

The viscosities of the three-layer cosmetics were measured by the following method. The shear viscosity of each cosmetic was measured at 25° C. using a rotating viscometer (Rheolab QC by Anton Paar GmbH) (rotational speed: 100 rpm).

The cosmetics of the Examples and Comparative Examples were evaluated for “separation”, “redispersibility”, “long-lasting moisturizing effect” and “smoothness”, on the following scales. The “long-lasting moisturizing effect” and “smoothness” parameters were evaluated by a single use test on skin by an evaluation panel of cosmetic experts.

### (1) Separation

After mixing by shaking followed by standing at room temperature, the outer appearance was visually evaluated.
A: The interface between a uniform upper layer and a uniform middle layer and between a uniform middle layer and a uniform lower layer was clearly observable after standing for half a day
B: The interface between a uniform upper layer and a uniform middle layer and between a uniform middle layer and a uniform lower layer was clearly observable after standing for a full day
C: The interface among the upper layer, the middle layer and the lower layer was observable but indistinct after shaking and standing for a full day, with non-uniformity of either the upper layer, middle layer or lower layer
D: No observable interface between the upper layer and the middle layer and between the middle layer and lower layer after shaking and standing for a full day

### (2) Redispersibility

The mixture was shaken and allowed to stand for 1 month at 25° C., and then shaken again.
A: Uniformly dispersed after shaking ≤10 times
B: Uniformly dispersed after shaking 10 to ≤20 times
C: Dispersed but non-uniform after ≥20 times
D: No redispersion
(3) Long-lasting moisturizing effect
A: Very notable long-lasting moisturizing effect
B: Notable long-lasting moisturizing effect
C: Virtually no notable long-lasting moisturizing effect
D: Absolutely no notable long-lasting moisturizing effect

### (4) Smoothness

A: Very notable smoothness
B: Notable smoothness
C: Virtually no notable smoothness
D: Absolutely no notable smoothness

TABLE 1

| | | Ex- | | | | |
|---|---|---|---|---|---|---|
| | | ample | Comp. Example | | | |
| | | 1 | 1 | 2 | 3 | 4 |
| Polysaccharide, | Agar (CS-7) | 0.5 | — | — | — | — |
| aqueous phase | (mass %) | | | | | |
| (invention), or | Bentonite (mass %) | — | 0.5 | 0.5 | — | — |
| powder phase | Kaolin (mass %) | — | 0.5 | 0.5 | — | — |
| (comparative) | Magnesium sulfate | — | 1 | 1 | — | — |
| | (mass %) | | | | | |
| | Butylene glycol | 5 | 5 | 5 | 5 | 5 |
| | (mass %) | | | | | |
| | Glycerin | 5 | 5 | 5 | 5 | 5 |
| | Phenoxyethanol | | | | | |
| | (preservative) | 1 | 1 | 1 | 1 | 1 |
| | (mass %) | | | | | |
| | Ethanol (mass %) | 5 | 5 | 5 | 5 | 5 |
| | PPG-6 decyl | — | 0.5 | 0.5 | — | — |
| | tetradeceth-20 | | | | | |
| | (NIKKOL PEN- | | | | | |
| | 4620, solubilizer) | | | | | |
| | (mass %) | | | | | |
| | Water | rem. | rem. | rem. | rem. | rem. |
| Oil (phase) | Triethylhexanoin | 5 | — | — | 5 | 1 |
| | (mass %) glyceryl tri | | | | | |
| | (2-ethylhexanoate) | | | | | |
| | Squalane (mass %) | 5 | — | — | 5 | 5 |
| | Dimethicone 50CS | — | — | — | — | 5 |
| | (mass %) | | | | | |
| Perfume (mass %) | | 0.1 | 0.1 | 0.1 | 0.1 | 0.1 |
| Total amount (mass %) | | 100 | 100 | 100 | 100 | 100 |
| | Viscosity (cP) | <10 | <10 | <10 | <10 | <10 |
| | Separation | A | B | C | B | B |
| | Redispersibility | A | B | C | B | B |
| | Evaluation | | | | | |
| | Long-lasting | A | D | D | C | C |
| | moisturizing effect | | | | | |
| | Smoothness | A | B | B | D | D |

The term ‘rem’. means ‘qs100’.

TABLE 2

| | | | Comp. | | | | | | |
|---|---|---|---|---|---|---|---|---|---|
| | | | Example | Example | | | | | |
| | | | 5 | 2 | 3 | 4 | 5 | 6 | 7 |
| Polysaccharide, | Agar (mass %) | CS-7 | — | 0.2 | 0.4 | 0.6 | — | — | — |
| aqueous phase | | YAWARA | — | — | — | — | — | — | — |
| | | CS-310 | — | — | — | — | — | — | 0.5 |
| | | CS-33 | — | — | — | — | — | — | — |
| | Carrageenan (mass %) | | — | — | — | — | 0.5 | — | — |
| | Gellan gum (mass %) | | — | — | — | — | — | 0.5 | — |
| | Calcium chloride (mass %) | | — | — | — | — | — | 1 | — |
| | Potassium chloride (mass %) | | — | — | — | — | 1 | — | — |
| | Butylene glycol (mass %) | | 5 | 5 | 5 | 5 | 5 | 5 | 5 |
| | Glycerin (mass %) | | 5 | 5 | 5 | 5 | 5 | 5 | 5 |
| | Phenoxyethanol (preservative) (mass %) | | 1 | 1 | 1 | 1 | 1 | 1 | 1 |
| | Sodium chloride (salting-out agent) (mass %) | | 1 | 1 | 1 | — | — | 1 | 1 |
| | Water | | rem. | rem. | rem. | rem. | rem. | rem. | rem. |
| | Ethanol (mass %) | | 5 | 5 | 5 | 5 | 5 | 5 | 5 |
| Oil phase | Isononyl isononanoate (mass %) | Polar oil | 5 | 5 | 5 | 5 | 5 | 5 | 5 |
| | Isododecane (mass %) | Non-polar | 5 | 5 | 5 | 5 | 5 | 5 | 5 |
| | | volatile oil | | | | | | | |
| | Hydrogenated polyisobutene | Non-polar oil | 5 | 5 | 5 | 5 | 5 | 5 | 5 |
| | (mass %) | | | | | | | | |

| Perfume (mass %) | | 0.1 | 0.1 | 0.1 | 0.1 | 0.1 | 0.1 | 0.1 |
|---|---|---|---|---|---|---|---|---|
| Total amount (mass %) | | 100 | 100 | 100 | 100 | 100 | 100 | 100 |
| Evaluation | Viscosity (cP) | <10 | <10 | <10 | <10 | <10 | <10 | <10 |
| | Separation | B | A | A | A | B | B | B |
| | Redispersibility | B | A | A | A | A | A | A |
| | Long-lasting moisturizing effect | C | A | A | A | A | A | A |
| | Smoothness | D | B | B | A | B | B | A |

| | Comp. | | | | |
|---|---|---|---|---|---|
| | Example | Example | | | |
| | 6 | 8 | 9 | 10 | 11 |
| Polysaccharide, | 0.5 | 0.5 | — | — | — |
| aqueous phase | — | — | 0.5 | — | — |
| | — | — | — | 0.5 | — |
| | — | — | — | — | 0.5 |
| | — | | — | — | — |
| | — | | — | | — |
| | — | | — | — | — |
| | — | | — | — | — |
| | 5 | 5 | 5 | 5 | 5 |
| | 5 | 5 | 5 | 5 | 5 |
| | 1 | 1 | 1 | 1 | 1 |
| | 1 | 1 | 1 | 1 | 1 |
| | rem. | rem. | rem. | rem. | rem. |
| | 5 | 5 | 5 | 5 | 5 |
| Oil phase | 5 | 5 | 5 | 5 | 5 |
| | 5 | 5 | 5 | 5 | 5 |
| | 5 | 5 | 5 | 5 | 5 |
| Perfume (mass %) | 0.1 | 0.1 | 0.1 | 0.1 | 0.1 |
| Total amount (mass %) | 100 | 100 | 100 | 100 | 100 |
| Evaluation | >200 | <10 | <10 | <10 | <10 |
| | D | A | B | B | A |
| | D | A | A | A | A |
| | B | A | A | A | A |
| | C | A | B | A | B |

TABLE 3

| | | | Example | | | | | | | | | |
|---|---|---|---|---|---|---|---|---|---|---|---|---|
| | | | 12 | 13 | 14 | 15 | 16 | 17 | 18 | 19 | 20 | 21 |
| Polysaccharide, | Agar (mass %) | CS-7 | 0.5 | 0.5 | 0.5 | 0.5 | 0.5 | 0.5 | 0.5 | 0.5 | 0.5 | 0.5 |
| Aqueous | Butylene glycol (mass %) | | 5 | 5 | 5 | 5 | 5 | 5 | 5 | 5 | 5 | 5 |
| phase | Glycerin (mass %) | | 5 | 5 | 5 | 5 | 5 | 5 | 5 | 5 | 5 | 5 |
| | Phenoxyethanol (preservative) (mass %) | | 1 | 1 | 1 | 1 | 1 | 1 | 1 | 1 | 1 | 1 |
| | Sodium chloride (salting-out agent) (mass %) | | 1 | 1 | — | — | 1 | 1 | 1 | 1 | 1 | 1 |
| | Water | | rem. | rem. | rem. | rem. | rem. | rem. | rem. | rem. | rem. | rem. |
| | Ethanol (mass %) | | 5 | 5 | 5 | 5 | 5 | 5 | 5 | 5 | 5 | 5 |
| Oil phase | Isononyl isononanoate (mass %) | Polar oil | 5 | 15 | — | — | — | — | — | — | — | — |
| | Isododecane (mass %) | Non-polar volatile oil | 10 | — | 15 | — | — | — | — | — | — | — |
| | Methyltrimethicone (mass %) | Volatile silicone oil | — | — | — | 15 | — | — | — | — | — | — |
| | Cyclopentasiloxane 20CS | Volatile silicone oil | — | — | — | — | 15 | — | — | — | — | — |
| | (mass %) | | | | | | | | | | | |
| | Dimethicone (mass %) | silicone oil | — | — | — | — | — | 15 | — | — | — | — |
| | Triethylhexanoin (mass %) | Polar oil | — | — | — | — | — | — | 15 | — | — | — |
| | Squalane (mass %) | Non-polar oil | — | — | — | — | — | — | — | 15 | — | — |
| | Jojoba seed oil (mass %) | Polar oil | — | — | — | — | — | — | — | — | 15 | — |
| | Hydrogenated polyisobutene | Non-polar oil | — | — | — | — | — | — | — | — | — | 15 |
| | (mass %) | | | | | | | | | | | |

| Perfume (mass %) | | 0.1 | 0.1 | 0.1 | 0.1 | 0.1 | 0.1 | 0.1 | 0.1 | 0.1 | 0.1 |
|---|---|---|---|---|---|---|---|---|---|---|---|
| Total amount (mass %) | | 100 | 100 | 100 | 100 | 100 | 100 | 100 | 100 | 100 | 100 |
| Evaluation | Viscosity (cP) | <10 | <10 | <10 | <10 | <10 | <10 | <10 | <10 | <10 | <10 |
| | Separation | A | A | A | A | A | A | B | B | B | A |
| | Redispersibility | A | A | A | A | A | A | A | A | A | A |
| | Long-lasting moisturizing effect | A | A | B | B | B | B | A | A | A | A |
| | Smoothness | A | A | A | A | A | B | B | B | B | A |

These results showed that the redispersible three-layer cosmetic comprising an aqueous phase, an oil phase and a polysaccharide gel particles having a mean particle size of 0.1 to 1000 μm according to the invention are well redispersible and have improved long-lasting moisturizing effect and smoothness in comparison to two-layer cosmetic (comparative example 1, 2, 3 and 4).

1. A redispersible three-layer cosmetic comprising an aqueous phase, an oil and a polysaccharide gel particles having a mean particle size of 0.1 to 1000 μm

2. The redispersible three-layer cosmetic according to claim 1, wherein the polysaccharide is selected from the group consisting of agar, carrageenan, gellan gum, sodium alginate, tamarind gum, mannan, locust bean gum, and mixtures thereof, in particular agar, carrageenan and gellan gum, and more preferably agar.

3. The redispersible three-layer cosmetic according to claim 1 or 2, wherein the viscosity of the three-layer cosmetic is 1 cP to 200 cP at 25° C.

4. The redispersible three-layer cosmetic according to any one of claims 1 to 3, wherein the aqueous phase comprises a polyol, in particular selected from the group consisting of diols, triols and mixtures thereof, in particular butylene glycol (1,3-butylene glycol), pentylene glycol, propanediol, dipropylene glycol, polyalkylene glycols, glycerin, and mixtures thereof.

5. The redispersible three-layer cosmetic according to any one of claims 1 to 4, wherein the aqueous phase comprises a mono-alcohol, in particular selected from the group consisting of ethanol, propanol, isopropanol and butanol, preferably ethanol.

6. The redispersible three-layer cosmetic according to any one of claims 1 to 5, wherein the aqueous phase comprises an aryloxyalkanol, preferably phenoxyethanol.

7. The redispersible three-layer cosmetic according to any one of claims 1 to 6, wherein the oil comprises two or more selected from the group consisting of polar oils, non-polar oils and silicone oils.

8. The redispersible three-layer cosmetic according to any one of claims 1 to 7, which is water product, a cleansing lotion, a face cleanser, an essence, a makeup base, a lotion mist, or a sunscreen or the like.

9. Cosmetic process for caring for and/or making-up keratinic materials comprising the application onto keratinic materials, in particular onto skin, of the redispersible three-layer cosmetic as defined in any one of the claims 1 to 8.

10. Cosmetic process according to claim 9, wherein the redispersible three-layer cosmetic is shaken before use.

11. Cosmetic process according to claim 9 or 10, wherein three-layer cosmetic provides a long-lasting moisturizing effect and smoothness onto keratinic materials, in particular onto skin, on which it is applied.
